(19) United States
(12) Patent Application Publication
(10) Pub. No.: US 2007/0116659 A1
(43) Pub. Date: May 24, 2007

(54) ELASTASE INHIBITOR

(75) Inventors: Shigeru MORIWAKI, Haga-gun (JP); Naoko Morisaki, Haga-gun (JP); Christine Hall, Cincinnati, OH (US)

Correspondence Address: OBLON, SPIVAK, MCCLELLAND, MAIER & NEUSTADT, P.C., 1940 DUKE STREET, ALEXANDRIA, VA, 22314, US

(73) Assignee: Kao Corporation, Tokyo (JP)

(21) Appl. No.: 11/627,704

(22) Filed: Jan. 26, 2007

Related U.S. Application Data
(63) Continuation of application No. 10/855,358, filed on May 28, 2004, now abandoned.

(51) Int. Cl. A61K 8/64

(52) U.S. Cl. 424/70.14; 424/73

(57) ABSTRACT

The present invention relates to an elastase inhibitor containing, as an active ingredient, a protein hydrolysate (with soybean protein hydrolysate and almond protein hydrolysate being excluded). According to the present invention, there can be provided pharmaceuticals and cosmetics which exhibit inhibitory effect against the growth of body hair, and skin aging preventing effect by restoring elasticity and tonicity of the skin and reducing wrinkles.

### FIELD OF THE INVENTION

The present invention relates to an elastase inhibitor.

### BACKGROUND OF THE INVENTION

Elastin is a structural protein found in elastic, resilient tissue constituting, for example, arteries, tendons, or the skin. Elastin molecules present in skin tissue mutually cross-link to thereby contribute to the elasticity of the tissue. However, when over-expression of elastase, which is an elastin-degrading enzyme, occurs due to UV exposure or aging, elastin is denatured or degraded, whereby elasticity of skin is considered to be declined. Meanwhile, in recent years, elastase activity in skin tissue has been reported to show good correspondence to the hair cycle; specifically, in the follicle proliferative stage (growth stage) elastase activity rises, and in the regression stage or resting stage the activity decreases, proving that an increase of elastase activity in the skin tissue is essential to the development and growth of follicles (see Japanese Patent Application Laid-Open (kokai) No. 11-199450).

Therefore, inhibition of the activity of elastase in skin tissue would be significant in that elasticity and tonicity would be imparted to the skin, and in the growth of hair suppressed.

Biologically, the hair on the head or body hair protects important parts of the body, including the head, chest, and limbs. However, such a function of the hair for protecting body parts has diminished as a variety of protective means such as clothing and protectors have emerged and been developed. Also, in recent years, from the esthetic point of view, people have come to favor hairless skin, particularly on the limbs. To meet such a trend, there is a need for the development of a hair growth inhibitor with which hair removal treatment can be performed at a lower frequency.

Meanwhile, hydrolysates of milk protein (casein and whey protein), maize-derived protein, sunflower-derived protein, fish-derived protein, or a like protein have been reported to exhibit action of inhibiting angiotensin-I-converting enzyme; hydrolysates of wheat gluten or casein have been reported to exhibit analgesic action; hydrolysates of rice protein or soybean protein have been reported to exhibit immunomodulatory action; and milk protein hydrolysate has been reported to exhibit protective action against peroxidation of essential fatty acids (see Kitts D D, Weiler K, Curr. Pharm. Des. 2003; 9(16): 1309-23).

However, it remains unknown that such a protein hydrolysate exhibits elastase inhibitory action.

### SUMMARY OF THE INVENTION

The present invention is directed to provision of pharmaceuticals or cosmetic compositions which exert, through inhibition of elastase activity, a body hair growth suppressing effect and effects of retarding aging of the skin, inter alia, restoring elasticity and tonicity of the skin and reducing wrinkles.

Accordingly, the present invention provides an elastase inhibitor containing, as an active ingredient, a protein hydrolysate other than soybean protein hydrolysate or almond protein hydrolysate.

The present invention also provides a hair growth inhibitor and a skin aging preventing agent, containing, as an active ingredient, a protein hydrolysate other than soybean protein hydrolysate or almond protein hydrolysate.

The present invention also provides a method for inhibiting growth of hair or a method for preventing aging of the skin, characterized by administering, to a subject in need thereof, an effective amount of a protein hydrolysate other than soybean protein hydrolysate or almond protein hydrolysate.

### DETAILED DESCRIPTION OF THE INVENTION

The present inventors have searched for a natural material that exhibits an elastase inhibitory activity and is very safe, and have found that a variety of protein hydrolysates exhibit elastase inhibitory action and are useful as pharmaceutical compositions or cosmetic compositions for suppressing growth of hair or preventing aging of the skin.

The protein hydrolysate of the present invention exhibits an elastase inhibitory action and is very safe, and therefore, the hydrolysate is very useful as a pharmaceutical composition or a cosmetic composition which exhibits an effect of preventing hair growth and an effect of preventing skin aging and which meets high safety standards. Use of the protein hydrolysate of the present invention enables reduction in frequency of body hair removal treatment; for example, epilation or shaving by use of a razor.

As used herein, the elastase inhibitor refers to as a material capable of inhibiting degradative activity of elastase (which is an enzyme causing degradation of elastin). In particular, elastase inhibitors that function to specifically inhibit fibroblast-derived elastase are preferred. For example, the elastase inhibitor may be a material that exhibits enzyme-inhibitory activity in an enzyme activity assay system employing, as a substrate, 125 mM N-Suc-(Ala)₃-p-nitroanillide. In the present specification, the term “enzyme inhibitor” means a material which functions to prevent catalytic action through, for example, establishing bonding with an enzyme, and does not include a material which induces protein denaturation to thereby cause deactivation of the enzyme. In particular, when the elastase inhibitor of the present invention is used in an amount of 0.03 wt. % (as reduced to solid content), from the viewpoint of effect obtained, the inhibitor preferably has an enzyme inhibitory activity of 10% or more, more preferably 30% or more, even more preferably 50% or more, and most preferably 70% or more.

Examples of the protein hydrolysates of the present invention include those hydrolysates obtained through hydrolysis of a protein-containing raw material (with soybeans and almonds being excluded) with an enzyme, an acid, or an alkali. In particular, those obtained by hydrolysis with an enzyme are preferred, from the viewpoint of elastase inhibitory effect.

Examples of the protein-containing raw material include, but are not limited to, milk, silk, wheat, maize, rice, pea, oat, sunflower, fish, seaweeds, casein, collagen, elastin, and keratin. Of these, preferred examples are milk, silk, wheat, rice, pea, oat, collagen, elastin, and keratin.

The resultant protein hydrolysates preferably have an average molecular weight of 10,000 or less, more preferably 4,000 or less.

Hydrolysis by use of an enzyme is typically performed by preparing a solution containing a protein in an amount of 1 to 35 wt. %, preferably 5 to 15 wt. %, and causing an enzyme to react therewith in a working pH range and working temperature range of the employed enzyme.

No particular limitations are imposed on the enzyme to be used for effecting hydrolysis, so long as the protein of interest can be degraded. For example, exoprotease or endoprotease may be used. Moreover, any enzyme of animal origin, plant origin, or microorganism origin may be used. Specific examples of such enzymes include, but are not limited to, serine proteases such as trypsin, chymotrypsin, and carboxypeptidase; thiol proteases such as papain, ficin, and bromelain; and carboxyproteases such as pepsin.

The treatment time during which enzymatic degradation takes place differs depending on the activity and amount of the protease employed. Generally, the treatment time is about 5 minutes to 30 hours, preferably about 30 minutes to 10 hours.

Hydrolyzing treatment through use of an acid or alkali may be performed, for example, by immersing, generally at a temperature of 10 to 80° C., a protein-containing raw material in a mixture of one or more species selected from among water and/or methanol, ethanol, propanol, butanol, propylene glycol, and 1,3-butylene glycol, preferably in a mixture prepared by adding, to water and/or ethanol, an acid such as 1 to 30 vol. % sulfuric acid, hydrochloric acid, acetic acid, or phosphoric acid, or an alkali such as 0.1 to 10 N sodium hydroxide, or potassium hydroxide.

The hydrolysate resulting from the above-described hydrolyzing treatment may be employed after, if necessary, purification through centrifugal separation, salting out, or filtration, followed by drying.

Alternatively, a commercially available protein hydrolysate may be used as is or after suitable purification. Examples of such commercial products include, but are not limited to, silk protein hydrolysate (Solu-Silk Protein; Arch), milk protein hydrolysate (Hydromilk Protein EN-20; Arch), rice protein hydrolysate (Rice-Pro-Tein BK; Maybrook), wheat protein hydrolysate (Wheat-Tein NL; Maybrook), pea protein hydrolysate (Pea Pro-Tein BK; Maybrook), keratin hydrolysate (Kera-Tein 1000; Maybrook), collagen hydrolysate (Cosmetic N-55; Maybrook), and oat protein hydrolysate (Cosmoist 0-25; Croda).

As will be described hereinbelow, any of the thus-obtained protein hydrolysates is endowed with elastase inhibitory activity. Therefore, formulations containing, as an active ingredient, the protein hydrolysate of the present invention are effective for producing cosmetic compositions, drug compositions, or quasi-drug compositions, which exhibit skin care effect such as hair growth inhibitory effect or skin aging preventive effect such as mitigation of wrinkles.

The elastase inhibitor, hair growth inhibitor, and skin aging preventing agent of the present invention are preferably processed into dermal medicines for external use. For example, they may take the form of water-in-oil emulsion or oil-in-water emulsion, cream, lotion, gel, foam, essence, foundation, pack, stick, or powder.

In particular, a hair growth inhibitor is preferably formed into a cosmetic composition for use in a process for eliminating body hair, such as body hair removal, epilation, or shaving. Specifically, preferred examples of such products include hair removers in the form of paste, cream, aerosol, soap, etc.; depilatories in the form of wax, gel, sheet, etc.; post-treatment agents to be used after hair removal treatment or epilation treatment, in the form of lotion, cream, etc.; antiperspirants and deodorant cosmetic compositions such as deodorant lotion, deodorant powder, deodorant spray, and deodorant stick; pre-shaving treatment agents such as preshave lotions; shaving cosmetic compositions such as shaving creams; or post-shaving treatment agents such as aftershave lotions.

Any of the protein hydrolysates is incorporated into the elastase inhibitor, the hair growth inhibitor, or the skin aging preventing agent of the present invention in such an amount that is effective for securing the hair growth inhibitory effect and skin aging preventing effect. The amount is 0.00001 to 50 wt. %, preferably 0.1 to 10 wt. %, more preferably 0.5 to 10 wt. %. In particular, in order to obtain the maximum hair growth inhibitory effect, the protein hydrolysate of the present invention is preferably incorporated in an amount of 0.1 wt. % or more, more preferably 0.5 wt. % or more.

The hair growth inhibitor and the skin aging preventing agent of the present invention may be combined with a variety of ingredients that are usually employed for formulating end products. Examples of such ingredients falling within the category of typical cosmetic ingredients include oils, surfactants, purified water, alcohols, chelating agents, pH modifiers, antiseptics, thickeners, emulsifiers, emulsion stabilizers, pigments, and perfumes. Examples further include UV absorbers, whitening agents, humectants, sebum secretion suppressors, softening agents, keratin protective agents, medicinal agents, antioxidants, and solvents. These may be arbitrarily selected and combined, to thereby formulate cosmetics, drugs for external use, or quasi-drugs.

To the hair growth inhibitor in particular, in accordance with needs, there may be added a keratin dissolver and an ingredient exhibiting hair-growth suppressing effect or hair removal effect, such as thioglycolic acid or a salt thereof. Examples of the keratin dissolver include, but are not limited to, lactic acid, bioprase, salicylic acid, glycolic acid, citric acid, and malic acid. Examples of salts of thioglycolic acid include, but are not limited to, sodium salt, potassium salt, ammonium salt, and alkanolamine salts, where the alkanolamine may be, for example, monoethanolamine, diethanolamine, or triethanolamine. Any of these keratin dissolvers, thioglycolic acid, and thioglycolic acid salts is incorporated preferably in an amount of 0.01 to 10 wt. %, more preferably 0.05 to 5 wt. %.

### EXAMPLES

### Example 1

### Elastase Activity Inhibition Test

Fibroblasts derived from healty human commercially available from Dainippon Pharmaceutical Co., Ltd. were subcultured in a DME medium supplemented with 10% fetal calf serum, and employed in the test. Cells scraped with a rubber policeman from the Petri dish were suspended in saline, and then collected by use of a low-speed centrifugal separator. The cells were washed three times with saline. The cells were suspended in 0.1% Triton X-100/0.2 M Tris-HCl buffer (pH 8.0) and then subjected to ultrasonication treatment, whereby an enzyme liquid was obtained. The substrate employed in the enzyme activity assay is 125mM N-Suc-(Ala)3-p-nitroanillide. Each of the protein hydrolysates shown in Table 1 was added to the enzyme liquid so as to attain a final concentration of 1%, and reaction was allowed to proceed for one hour at 37° C. The reaction was terminated by the addition of 5 μL acetic acid. Absorbance at 405 nm was determined by use of a spectrophotometer, whereby the amount of produced p-nitroaniline was calculated. Elastase activity inhibition (%) was calculated from the below-described equation. The results confirmed that the protein hydrolysates exhibit excellent elastase activity inhibitory effect (Table 1).

Elastase activity inhibition (%)=100−(amount of p-nitroaniline as determined when protein hydrolysate was added)/(amount of p-nitroaniline as determined when protein hydrolysate was not added)×100 (Left blank)

TABLE 1

| | | | Elastase activity |
|---|---|---|---|
| Protein hydrolysate | | | inhibition |
| | Product name | Source | (%) |
| Milk protein | Hydromilk Protein | Arch | 92.03 |
| | EN-20 | | |
| Silk protein | Solu-Silk Protein | Arch | 53.67 |
| Wheat protein | Wheat-Tein NL | Maybrook | 92.10 |
| Elastin | Crolastin | Croda | 73.85 |
| Rice protein | Rice Pro-Tein Bk | Maybrook | 97.55 |
| Pea protein | Pea Pro-Tein BK | Maybrook | 95.19 |
| Oat protein | Cosmoist 0-25 | Croda | 92.85 |
| Collagen | Cosmetic N-55 | Maybrook | 97.46 |
| Keratin | Kera-Tein 1000 | Maybrook | 82.02 |

1-8. (canceled)

9. A protein hydrolysate, other than almond protein hydrolysate or soybean protein hydrolysate, which inhibits elastase.

10. A method for inhibiting the growth of hair comprising:
administering to a subject in need thereof an effective amount of a protein hydrolysate having an average molecular weight of 10,000 or less, wherein said protein hydrolysate is not a soybean protein hydrolysate or an almond protein hydrolysate.

11. The method for inhibiting growth of hair of claim 10, wherein the protein hydrolysate is selected from the group consisting of milk protein, silk protein, rice protein, pea protein, collagen, elastin, and keratin.

12. The method of claim 10, wherein said protein hydrolysate is wheat protein hydrolysate.

13. The method of claim 10, wherein said protein hydrolysate is oat protein hydrolysate.

14. The method for inhibiting growth of hair of claim 10, wherein the protein hydrolysate has an average molecular weight of 4,000 or less.

15. The method of claim 10 comprising:
externally administering to the skin of a subject in need thereof an amount of a protein hydrolysate having an average molecular weight of 10,000 or less sufficient to inhibit elastase activity in said subject's skin and suppress hair growth.

16. A method for treating aging skin comprising:
externally administering to the skin of a subject in need thereof an amount of a protein hydrolysate having an average molecular weight of 10,000 or less sufficient to inhibit elastase activity in said subject's skin,
wherein said protein hydrolysate is not a soybean protein hydrolysate or an almond protein hydrolysate.

17. The protein hydrolysate of claim 9, which is produced by enzymatic hydrolysis.

18. The protein hydrolysate of claim 9, which is produced by acid hydrolysis.

19. The protein hydrolysate of claim 9, which is produced by alkali hydrolysis.

20. The protein hydrolysate of claim 9, further comprising a keratin dissolver selected from the group consisting of lactic acid, bioprase, salicylic acid, glycolic acid, citric acid, and malic acid; or mixtures thereof.

21. The protein hydrolysate of claim 9, further comprising a thioglycolic acid or a salt thereof.
